(19) United States
(12) Patent Application Publication
(10) Pub. No.: US 2013/0304028 A1
(43) Pub. Date: Nov. 14, 2013

(54) CATHETER CLAMPS AND CATHETERS

(71) Applicants: Tariq Chaudhry, Tampa, FL (US); Scott Hampton, Gainesville, GA (US)

(72) Inventors: Tariq Chaudhry, Tampa, FL (US); Scott Hampton, Gainesville, GA (US)

(73) Assignee: H. Lee Moffitt Cancer Center and Research Institute, Inc., Tampa, FL (US)

(21) Appl. No.: 13/983,392

(22) PCT Filed: Feb. 3, 2012
(86) PCT No.: PCT/US12/23821

§ 371 Date: Aug. 2, 2013

Related U.S. Application Data
(60) Provisional application No. 61/439,179, filed on Feb. 3, 2011.
(60) Provisional application No. 61/550,532, filed on Oct. 24, 2011.

(51) Int. Cl. A61M 39/28

(52) U.S. Cl. CPC A61M 39/28
USPC 604/508; 604/250

(57) ABSTRACT

Provided are devices and methods for reducing or preventing fluid flow through a catheter lumen. For example, the devices and methods can be used to reduce blood flow through a catheter. Optionally, the catheter is an intravenous catheter. Also provided are catheters.

### CROSS REFERENCE TO RELATED APPLICATIONS

This application claims the benefit of U.S. Provisional Application Nos. 61/439,179, filed on Feb. 3, 2011, and 61/550,532, filed on Oct. 24, 2011, which are incorporated by reference herein in their entirety.

### TECHNICAL FIELD

The present application relates to catheters and to devices and methods for reducing or preventing fluid flow through a catheter.

### BACKGROUND

Hospital acquired infections can result from inadvertent spread of contaminants by hospital staff taking care of patients. Such infections lead to unanticipated prolonged hospital stays and increase healthcare costs in the form of expensive treatments.

Intravenous catheter placement carries a significant exposure risk of patient's blood to the hospital staff During the placement of an intravenous catheter, it is common for the patient's blood to leak out through the catheter end after a vein has been accessed and before the IV line is connected to the catheter. In addition to contaminating the patient's skin and the nurse's gloves, this creates an unpleasant sight for the patient. The nurse then connects the IV tubing and adjusts the flow-dial, thus inadvertently contaminating additional surfaces. During the hospital stay of a typical patient, multiple adjustments are made to the same intravenous line by additional hospital staff exposing even more personnel and surfaces to the patient's blood.

### SUMMARY

Provided are devices and methods for reducing or preventing fluid flow through a catheter lumen.

An example device comprises a first clamping surface and a second clamping surface opposed to the first clamping surface. The surfaces at least partially define a slot configured to accept a portion of the catheter through which fluid flow is to be reduced or prevented. The first and second surfaces are moveable relative to one another to narrow the slot and the narrowed slot at least partially occludes the catheter lumen to reduce or prevent fluid flow through the catheter.

Optionally, the catheter, or a portion thereof, is operatively located in a blood vessel of a subject. The blood vessel is optionally a vein or an artery. When the catheter is located in a blood vessel, the devices and methods can be used to prevent or reduce blood flow through the catheter.

When the catheter portion is positioned in the slot, it optionally has a needle threaded through its lumen. For example, a catheter with a needle threaded through its lumen may be positioned in a blood vessel of a subject. When positioned in the blood vessel, a portion of the catheter and needle is located in the lumen of the subject's blood vessel. The needle optionally prevents the first and second surfaces from moving relative to each other to narrow the slot. The needle can be withdrawn from the catheter lumen allowing the first and second surfaces to move relative to each other to narrow the slot and to at least partially occlude the catheter lumen upon removal of the needle from the catheter lumen.

Example devices can further comprise an actuator configured to move the surfaces relative to each other. Optionally, the actuator is a spring. For example, a first end of the spring is optionally coupled to the first surface and wherein a second end of the spring is optionally coupled to the second surface. A force generated by the spring causes movement of the surfaces relative to each other to narrow the slot and to at least partially occlude the lumen.

Further provided are catheters. The catheters can include a flexible tubular portion for placement into the vasculature of a subject and a clamping device having a first clamping surface and a second clamping surface opposed to the first clamping surface, wherein the surfaces at least partially define a space configured to accept a portion of the flexible tubular portions of the catheter. The first and second surfaces are moveable relative to one another to narrow the space. The narrowed space at least partially occludes the catheter lumen at a location along the tubular portion thereby reducing or preventing fluid flow through the catheter.

Optionally, the clamping device further comprises a first arm with a distal end connected to a first clamping body. The first clamping body optionally includes the first clamping surface. The clamping device optionally further comprises a second arm with a distal end connected to a second clamping body. The second clamping body optionally includes the second clamping surface.

The catheters optionally further include a hub portion that is located distal to the tubular portion. Each arm also has a proximal end that is connected to the hub portion. Optionally, the first clamping surface is located opposite the central midline axis of the catheter from the connection of the proximal end of the first arm with the hub. Optionally, the second clamping surface is located opposite the central midline axis of the catheter from the connection of the proximal end of the second arm with the hub. The arms bias the first and second clamping surfaces towards one another with sufficient force to deform the portion of the flexible tubular portion of the catheter that is positioned in the space between the clamping surfaces. The deformation of the flexible tubular portion is sufficient to reduce or eliminate blood flow through the catheter.

Example methods of reducing or preventing blood flow through a catheter located in a blood vessel of a subject are provided that comprise positioning the catheter having a needle threaded through its lumen in the slot of a device having a first clamping surface and a second clamping surface opposed to the first clamping surface. The surfaces at least partially define the slot in which the catheter is positioned. The needle is removed from the catheter lumen, allowing the first and second surfaces to move relative to each other to narrow the slot to at least partially occlude the catheter lumen, thereby reducing or preventing blood flow though the catheter. Optionally, the blood vessel is a vein or artery.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIGS. 1A and 1B are schematic illustrations of an intravenous catheter assembly.

FIGS. 2A and 2B are schematic illustrations of an example catheter clamping device.

FIG. 3 is a schematic illustration of an example catheter clamping device.

FIGS. 4A and 4B are schematic illustrations of an example catheter clamping device.

FIG. 5 is a schematic illustration of an example catheter clamping device.

FIG. 6 is a schematic illustration of an example catheter clamping device.

FIG. 7 is a schematic illustration of an example catheter clamping device.

FIGS. 8A-C are schematic illustrations of an example catheter clamping device.

FIGS. 9A-B are schematic illustrations of aspects of an example catheter clamping device.

FIGS. 10A-D are schematic illustrations of example catheter clamping devices.

FIGS. 11A-D are schematic illustrations of example catheter clamping devices.

FIGS. 12A-D are schematic illustrations of an example catheter clamping device.

FIGS. 13A-D are schematic illustrations of an example catheter.

### DETAILED DESCRIPTION

The present invention now will be described more fully hereinafter with reference to specific embodiments of the invention. The invention can be embodied in many different forms and should not be construed as limited to the embodiments set forth herein; rather, these embodiments are provided so that this disclosure will satisfy applicable legal requirements.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

As used throughout, by a “subject” is meant an individual. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. The term patient is used interchangeably with the term subject.

Provided are devices and methods for reducing or preventing fluid flow through a catheter lumen. For example, the devices and methods can be used to reduce blood flow through a catheter. Optionally, the catheter is an intravenous catheter. The fluid is not limited to blood and the catheter is not limited to an intravenous catheter. The clamping devices described are optionally applied to any type catheter, or any fluid line, to reduce or eliminate fluid flow therethrough the catheter or fluid line's lumen.

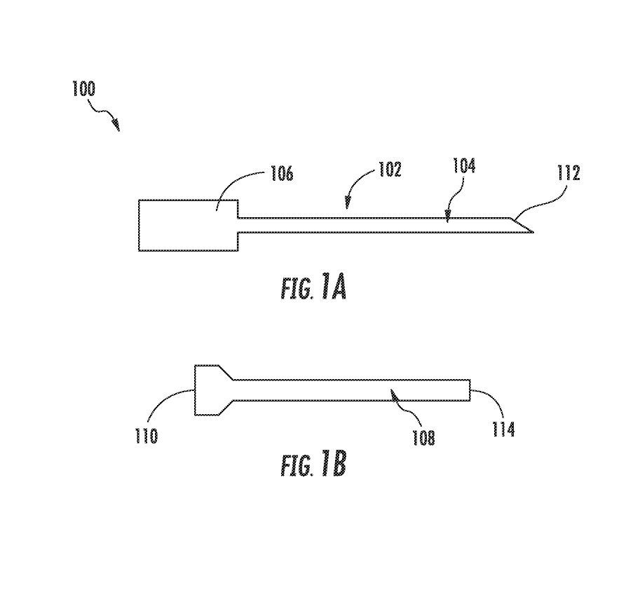

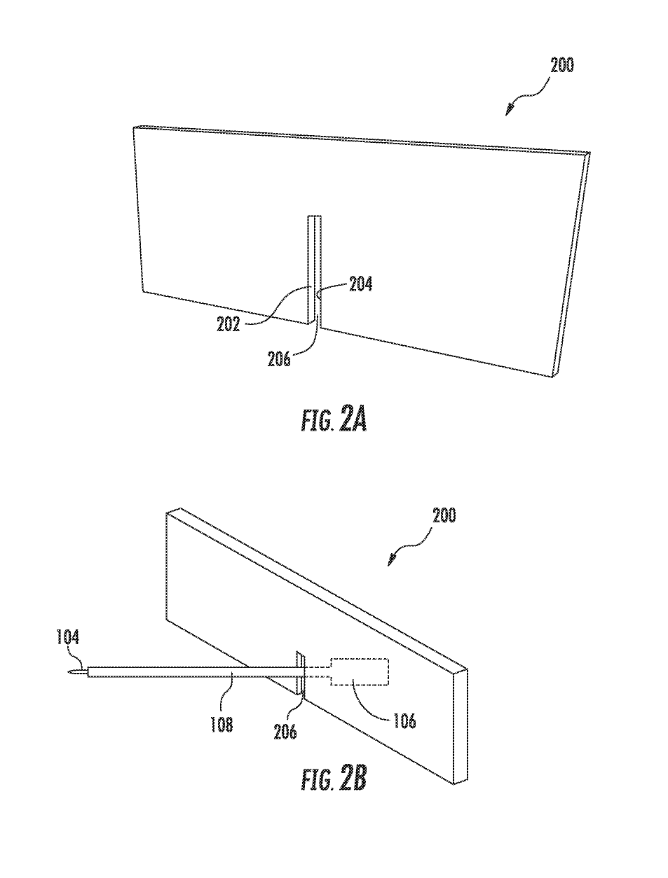

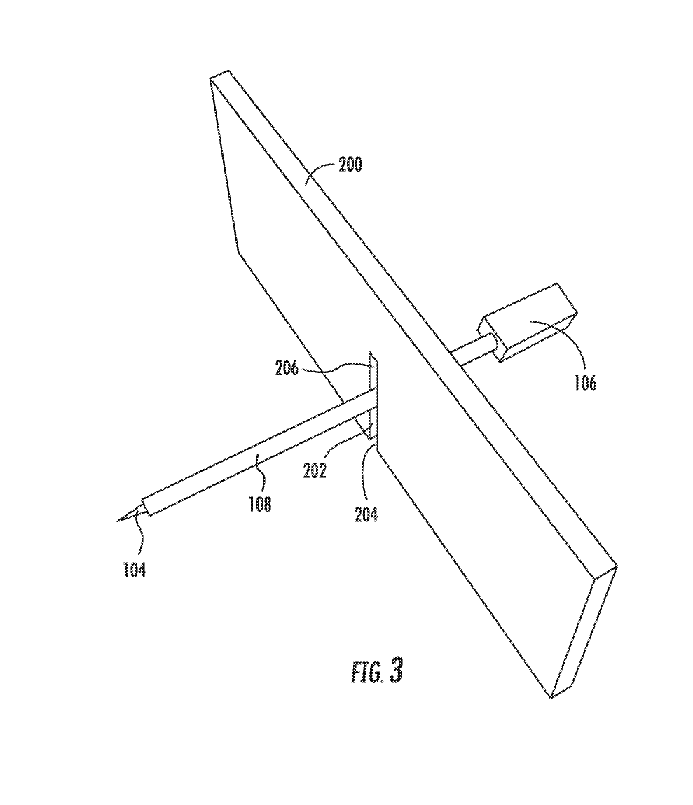

A typical intravenous catheter has two parts, a metal needle that punctures the vein and a plastic catheter which is left in the vein after the needle is pulled out. FIGS. 1A and 1B are schematic illustrations of a catheter 108 and a needle 102 for use in accessing the vascular system of a subject.

The needle shaft 104 can be threaded through the catheter 108 lumen until the hub 106 contacts a proximal portion 110 of the catheter. In this arrangement, the needle shaft is positioned in the catheter 108 lumen and the sharp tip 112 of the needle 102 protrudes at least partially from the catheter lumen at a distal tip 114 of the catheter.

The sharp tip 112 can pierce the vessel wall to allow the tip 112 and at least a portion of the needle and catheter to enter the vessel lumen. Once at least a portion of the needle 102 and catheter 108 are located in the vessel lumen, the needle can be removed by sliding the needle distally from the catheter while the catheter remains in the vessel lumen. These steps are common to the placement of catheters in the vascular system to gain access to the intravascular space. The catheter may then be used to deliver therapeutics, such as fluids, to the subject.

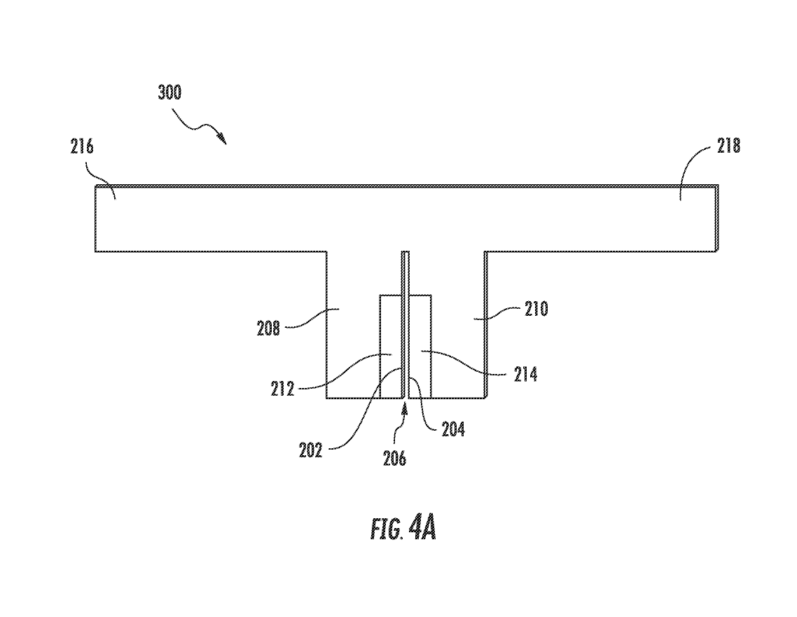

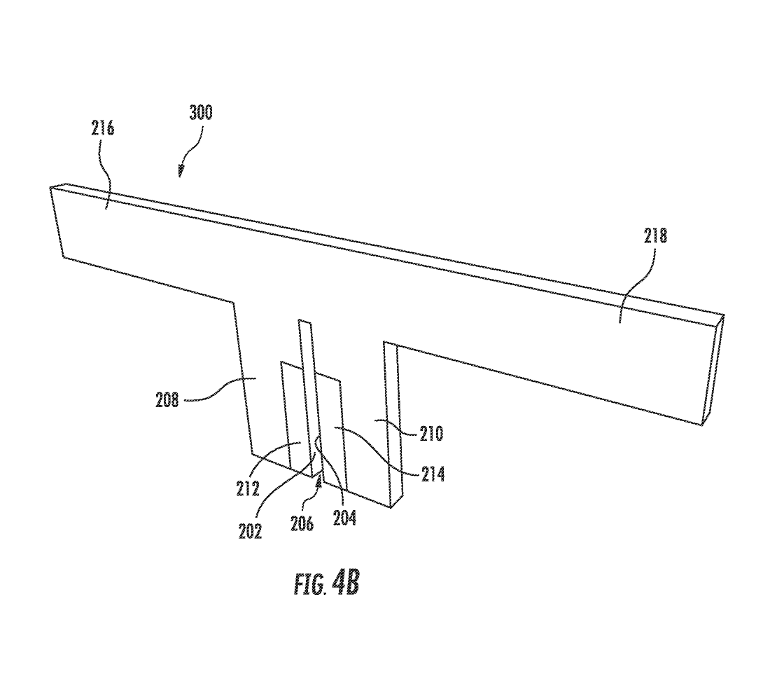

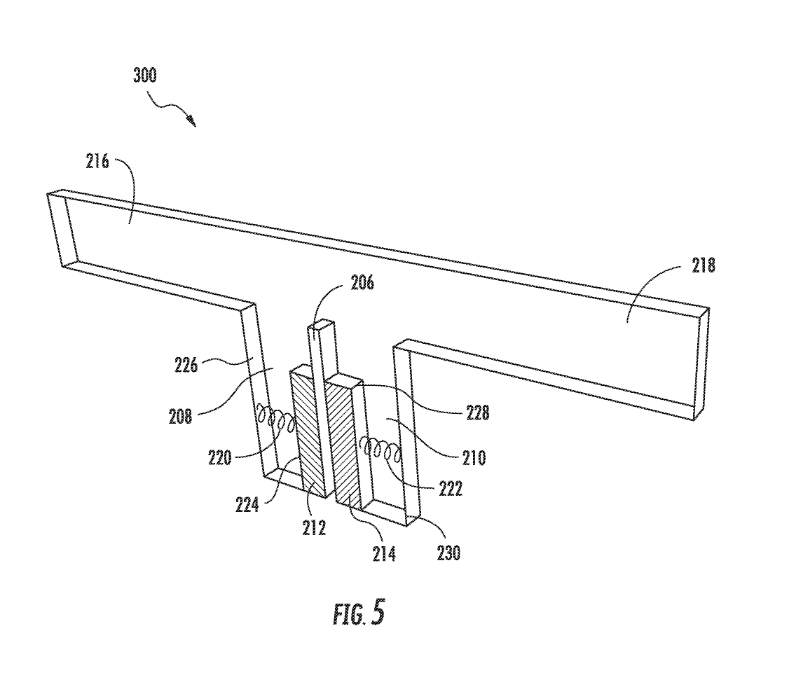

Referring now to FIGS. 2A and 2B, an example clamping device 200 is schematically illustrated. The device 200 comprises a first clamping surface 202 and a second clamping surface 204 that is opposed to the first clamping surface. The clamping surfaces (202 and 204) partially define a slot or space 206. The slot 206 is large enough in width to accept a portion of the catheter as shown in FIG. 2B. The catheter can be positioned in the slot 206 with the needle in its lumen as shown in FIG. 2B. In this arrangement, the needle in the catheter lumen can prevent the surfaces 202 and 204 from moving relative to each other to narrow the slot 206. FIG. 3 shows a perspective view of the catheter 108 and needle 104 positioned in the slot 206. In this configuration, the hub 106 of the needle prevents flow of blood out of the needle and blood flow is impeded through the catheter lumen by the needle itself which substantially fills the catheter lumen.

To gain access to the vascular space of the subject, the needle, including the hub, is retracted distally out of the vessel and out of the catheter lumen. Once removed, the proximal end 110 of the catheter 108 can be attached to a therapeutic agent for administration to the subject. For example, an intravenous fluid line can be placed in fluid communication with the lumen of the catheter after the needle and hub have been removed. In this way therapeutics, such as physiological fluids, can be administered through the catheter to the subject. Moreover the catheter can be used to remove blood from the subject.

When the needle 102, including the hub 106, is retraced from the catheter 108 the lumen of the catheter becomes patent to the flow of blood. Thus, intravascular, (IV or IA) catheter placement carries a significant exposure risk of patient's blood to the hospital staff. During the placement of an intravenous catheter, it is common for the patient's blood to leak out through the catheter end after a vein or artery has been accessed and before the IV or IA line is connected to the catheter. In addition to contaminating the patient's skin and the nurse's gloves, this creates an unpleasant sight for the patient. The nurse then connects the IV or IA tubing and adjusts the flow-dial thus inadvertently contaminating additional surfaces. During the hospital stay of a typical patient, multiple adjustments are made to the same intravenous line by additional hospital staff exposing even more personnel and surfaces to the patient's blood.

The clamping device 200, however, is used to prevent or reduce blood leakage from the catheter. In this regard, when the needle 102 is removed from the catheter, the opposed sides 202 and 204 move relative to one another to impede or prevent blood flow through the catheter by occluding or partially occluding the catheter lumen. Occluding includes narrowing or deforming the catheter lumen such that fluid flow is prevented or reduced through the catheter lumen. For example, a lumen's cross sectional shape can be narrowed or closed by moving the intraluminal walls closer together or into contact. Thus, a force is exerted on the catheter by the sides (202 and 204) that cause the lumen to narrow, or be crimped, between the sides as the slot 206 narrows. Optionally, the catheter lumen is partially narrowed, and blood spillage from the catheter is reduced or eliminated. Optionally, the catheter lumen is substantially closed and blood is prevented from flowing through the catheter.

The force may be exerted on the catheter from the sides (202 and 204) using a variety of mechanisms. For example, the material of the device may possess inherent tensile properties such that a natural force is exerted on the catheter and needle as they hold the slot 206 open. In this example, when the needle 102 is removed, the slot 206 automatically narrows as the material regains its natural resting position that includes a narrower slot 206. The devices described throughout may include such inherent tensile properties. Materials with such properties include, for example, plastics and polymeric compositions, as well as metals. Optionally, the devices described here can be sterilized, for example, by chemical and/or heat based techniques.

The slot 206 can be just wide enough to have a firm fit on the catheter 108. The needle 102 inside the catheter 108 acts as a stent during the venous access allowing the blood to flow inside the catheter hub to confirm the intravenous placement. As soon as the needle 102 is withdrawn from the catheter 108, the device puts a gentle squeeze on the catheter 108 preventing or reducing the blood from leaking out.

Another example device 300 is shown in FIGS. 4A, 4B and 5. As best shown in FIG. 5, the opposed surfaces 202 and 204 are located on the medial aspect of opposed movable units 212 and 214. The slot 206 is located between the surfaces and thus the surfaces (202 and 204) move relative to each other to narrow the slot 206. As the surfaces and units move relative to each other the slot is narrowed, which results in the occlusion or partial occlusion or narrowing the lumen of a catheter.

As further shown in FIG. 5, the moveable units may be actuated to move towards each other by springs 220 and 222, which force the units medially when the needle is removed from a catheter positioned in the slot as described in relation to the device 200 above.

Optionally, one of the units may remain stationary and one of the units may be actuated towards the stationary unit. In this case, the surfaces are still encouraged relative to each other to narrow the slot 206 and to thereby prevent or reduce blood flow through a catheter positioned in the slot. In addition, although coil springs are illustrated as providing the force to urge the surfaces to narrow the slot 206, other mechanisms can be used. For example, other spring types, such as, for example, torsion springs, tension springs and compression springs, micromotors, and the like, can be used to move the units and thus the surfaces (202 and 204) relative to each other.

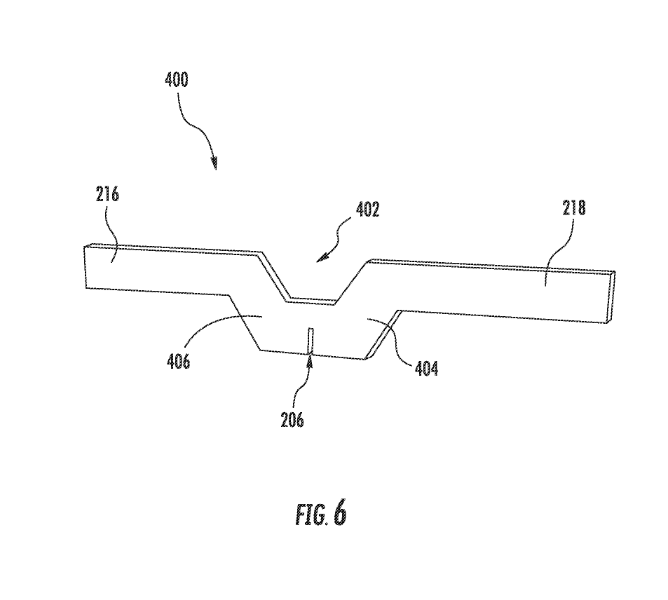

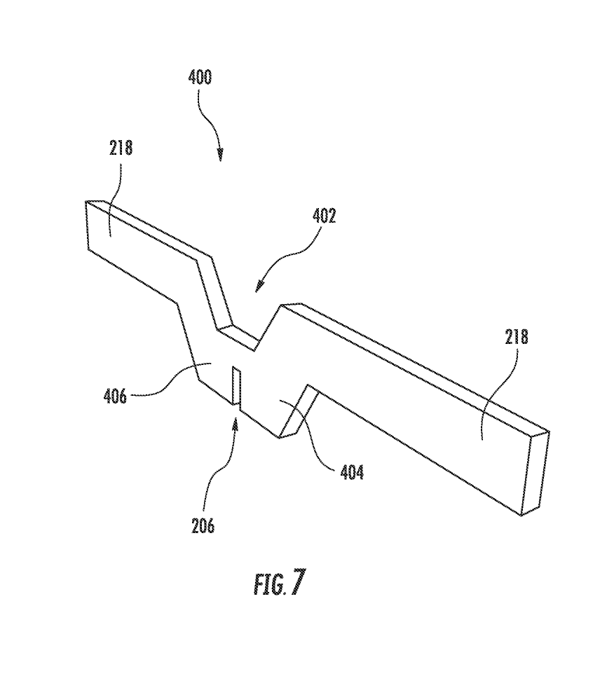

As also illustrated in FIGS. 4A, 4B and 5, the device 300 can include lateral wings 216 and 218. These wings may be used by an operator of the device 300 to position the catheter within the slot 206 while maintaining a sterile field. FIGS. 6 and 7 show perspective views of a device 400 with wings shaped to allow for easy location in an intravenous catheter kit and for easy positioning on and off of a catheter while maintaining a sterile environment during placement of the catheter.

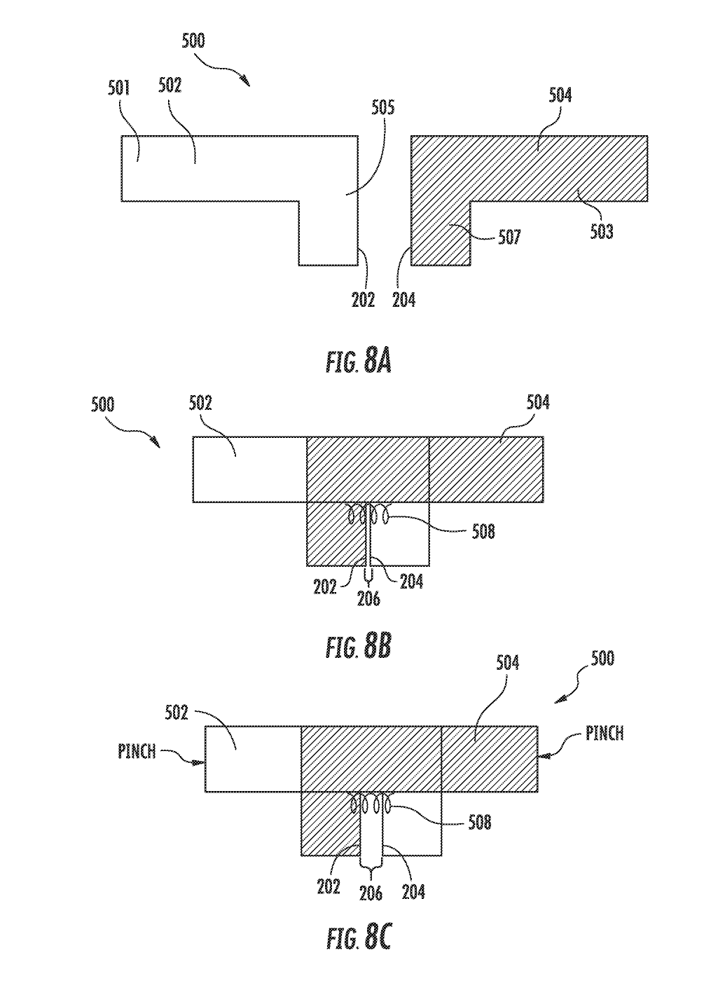

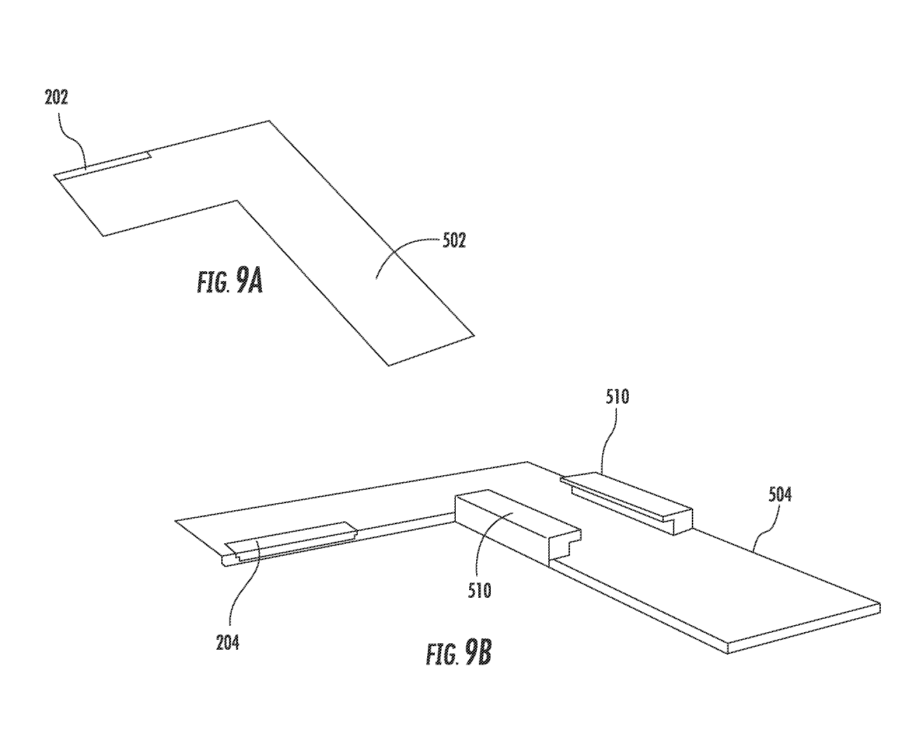

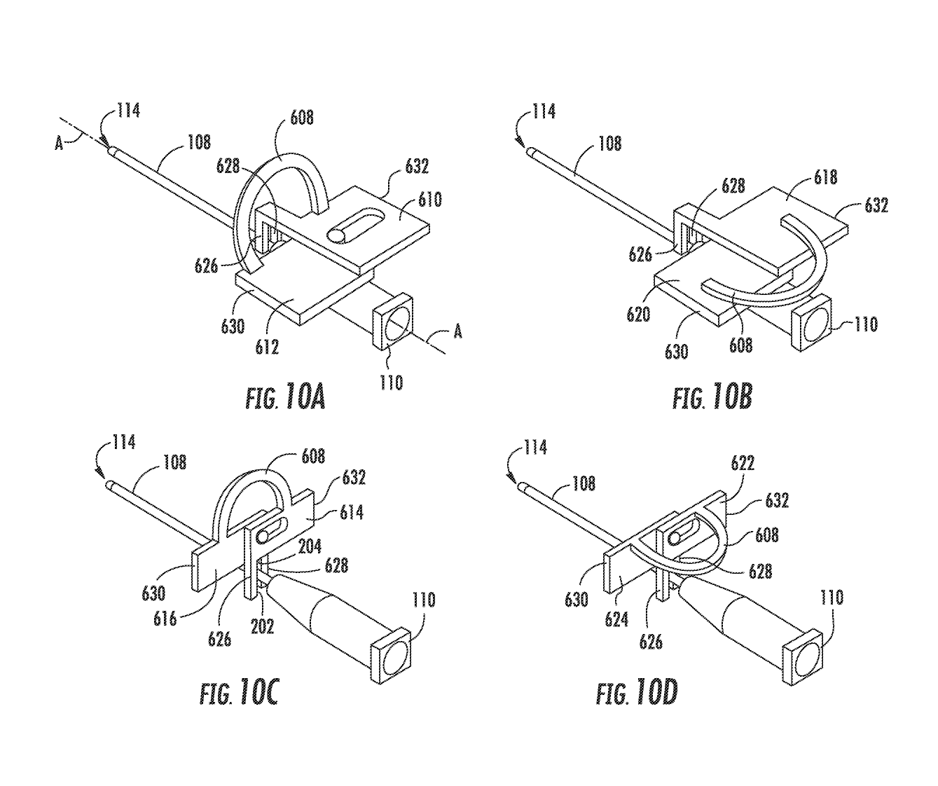

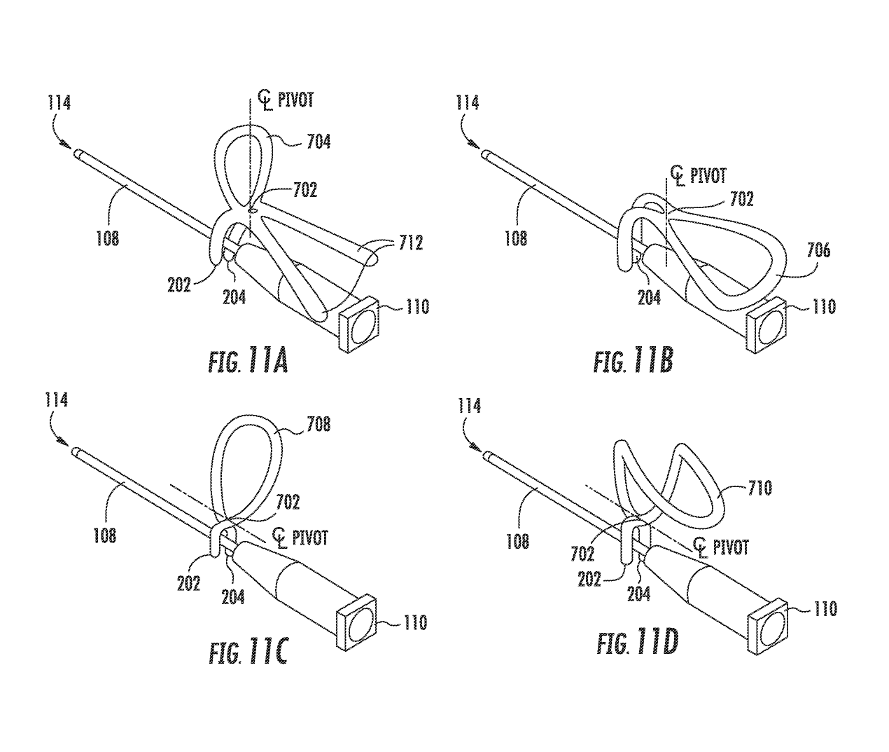

FIGS. 8A, B and C and FIGS. 9A and B, schematically illustrate a clamping device 500. The device includes two sliding units 502 and 504. Each sliding unit has horizontal portions 501 and 503 and vertical portions 505 and 507. The vertical portions 505 and 507 each have a medial surface that act as the opposed clamping surfaces (202 and 204) as described above.

As shown in FIG. 8B, in use, the sliding units overlap such that they can slide relative to one another. By sliding relative to one another, the slot 206 can widen or narrow depending on whether the surfaces 202 and 204 are moving towards each other (narrowing slot) or away from each other (widening slot). Optionally, the sliding units and, thus, the surfaces 202 and 204, are biased towards one another by an actuating device, such as a spring 508. In this regard, as shown in FIG. 8B, the spring 508 encourages the sliding units into a resting position where the slot is narrow enough to occlude, partially occlude, and/or reduce or prevent blood flow through a catheter positioned between the surfaces. Furthermore, as shown in FIG. 8C, the slot 206 can be widened by applying a force opposite to the force the spring applies to narrow the slot.

In operation, a force greater than the force of the spring 508 can first be applied to widen the slot 206 as shown in FIG. 8C. Then, a catheter 108 with a needle 102 in its lumen can be positioned in the slot between the surfaces 202 and 204. The widening force can then be removed or reduced to a level less that the opposite force of the spring 508 resulting in the relative movement of the sliding units 502 and 504. As the units slide relative to one another under the force of the spring, the slot 206 narrows until the narrowing is resisted and stopped by the catheter 108 with a needle 102 in its lumen.

If the distal tip of the catheter and needle are located in a vessel of a subject, the needle can be removed from the catheter. When the needle is removed, the sliding movement is no longer resisted by the needle/catheter combination and the slot 206 automatically narrows under the force of the spring to narrow or occlude the lumen of the catheter 108.

The slot 206 can narrow sufficiently to reduce or prevent spillage of blood from the catheter 108. While the blood flow is reduced or prevented from flowing through the catheter 108, an IV line, syringe, stopper, valve or other end unit may be operatively connected to the end 110 of the catheter. Once this operative connection is made, a force can be applied opposite and greater than the force of the spring 508 causing the slot 206 to widen and eventually release the catheter 108. Once released, the device 500 can be removed from the catheter 108 and the catheter can be used in typical medical operation.

FIGS. 9A and 9B schematically illustrate aspects of the sliding mechanism between the sliding unit 502 and the sliding unit 504. One of the sliding units comprises slots 510 that contain movement of the other sliding unit to a generally linear track as the units slide relative to one another. The mating of one of the sliding units within the slots also prevents the units from separating. FIGS. 9A and 9B also illustrate the surfaces 202 and 204, which function to impinge on the flow of blood through the catheter.

Additional example catheter clamping devices are illustrated in FIGS. 10A-10D. Each of these illustrations depicts a catheter clamp having first and second clamping surfaces that at least partially define a slot or space that is configured to accept a portion of the catheter 108. In each of these illustrations, the needle portion has been removed and the surfaces have moved relative to each other to clamp the catheter 108 to reduce or eliminate blood flow through the catheter lumen.

FIG. 10A illustrates a catheter clamping device comprising a first body portion 610 and a second body portion 612. The body portions (610 and 612) are each attached or integral with a force generating member 608. The body portions are configured to slide relative to one another under the force supplied by the force generating member 608. The force generation member naturally resists sliding of the body portions over each other towards a position in which the body portions are aligned with the first body portion directly over the second body portion.

The natural resistance of the force generating member 608 to such alignment, also results in production of a force that urges the body portions to slide across each other towards a non-aligned position such as shown in FIG. 10A. In this movement, the lateral edges (630 and 632) move away from the central longitudinal axis of the catheter 108 in opposing directions.

A first clamping portion 626 that is attached to or integral to the first body portion 610 is located on the opposite side of the central longitudinal axis (A-A) of the catheter from the body portion 610. The clamping portion located on the opposite side includes a clamping surface for clamping the catheter 108. A second clamping portion 628 that is attached or integral with the second body portion 612 is located on the opposite side of the central longitudinal axis of the catheter from the body portion 612. The clamping portion located on the opposite side includes a clamping surface for clamping the catheter 108.

The clamping surfaces are opposed to each other such that when the body portions slide relative to each other by the force of the force generating member, the opposed clamping surfaces move closer to each other to clamp down on the catheter. By applying a force that opposes and is greater than the force of the force generating member 608, the clamping surface can be separated as the body portions move back closer to a fully aligned position, wherein the fully aligned position refers to one being on top of the other.

FIG. 10B illustrates a catheter clamping device comprising a first body portion 618 and a second body portion 620. The body portions (618 and 620) are each attached or integral with a force generating member 608. The body portions are configured to slide relative to one another under the force supplied by the force generating member 608. The force generation member naturally resists sliding of the body portions over each other towards a position in which the body portions are fully aligned with the first body portion directly over the second body portion.

The natural resistance of the force generating member 608 to such alignment, also results in production of a force that urges the body portions to slide across each other towards a non-aligned position such as shown in FIG. 10B. In this movement, the lateral edges (630 and 632) move away from the central longitudinal axis of the catheter 108 in opposing directions.

A first clamping portion 626 that is attached to or integral to the first body portion 618 is located on the opposite side of the central longitudinal axis of the catheter from the body portion 618. The clamping portion located on the opposite side includes a clamping surface for clamping the catheter 108. A second clamping portion 628 that is attached or integral with the second body portion 620 is located on the opposite side of the central longitudinal axis of the catheter from the body portion 620. The clamping portion located on the opposite side includes a clamping surface for clamping the catheter 108.

The clamping surfaces are opposed to each other such that when the body portions (618 and 620) slide relative to each other by the force of the force generating member 608, the opposed clamping surfaces move closer to each other to clamp down on the catheter. By applying a force that opposes and is greater than the force of the force generating member 608, the clamping surface can be separated as the body portions move back closer to a fully aligned position, wherein the fully aligned position refers to one being on top of the other.

FIG. 10C illustrates a catheter clamping device comprising a first body portion 614 and a second body portion 616. The body portions (614 and 616) are each attached or integral with a force generating member 608. The body portions are configured to slide relative to one another under the force supplied by the force generating member 608. The force generation member naturally resists sliding of the body portions over each other towards a position in which the body portions are fully aligned with the first body 614 portion directly distal the second body portion 616.

The natural resistance of the force generating member 608 to such alignment, also results in production of a force that urges the body portions to slide across each other towards a non-aligned position such as shown in FIG. 10C. In this movement, the lateral edges (630 and 632) move away from the central longitudinal axis of the catheter 108 in opposing directions.

A first clamping portion 626 attached to or integral to the first body portion 614 is located on the opposite side of the central longitudinal axis of the catheter from the body portion 614. The clamping portion located on the opposite side includes a clamping surface for clamping the catheter 108. A second clamping portion 628 attached or integral with the second body portion 616 is located on the opposite side of the central longitudinal axis of the catheter from the body portion 616. The clamping portion located on the opposite side includes a clamping surface for clamping the catheter 108.

The clamping surfaces are opposed to each other such that when the body portions (614 and 616) slide relative to each other by the force of the force generating member 608, the opposed clamping surfaces move closer to each other to clamp down on the catheter 108. By applying a force that opposes and is greater than the force of the force generating member 608 the clamping surface can be separated as the body portions move back closer to a fully aligned position, wherein the fully aligned position refers to one being on top of the other.

FIG. 10D illustrates a catheter clamping device comprising a first body portion 622 and a second body portion 624. The body portions (622 and 624) are each attached or integral with a force generating member 608. The body portions are configured to slide relative to one another under the force supplied by the force generating member 608. The force generation member naturally resists sliding of the body portions over each other towards a position in which the body portions are fully aligned with the first body portion directly distal to the second body portion.

The natural resistance of the force generating member 608 to such alignment, also results in production of a force that urges the body portions to slide across each other towards a non-aligned position such as shown in FIG. 10D. In this movement, the lateral edges (630 and 632) move away from the central longitudinal axis of the catheter 108 in opposing directions.

A first clamping portion 626 attached to or integral to the first body portion 622 is located on the opposite side of the central longitudinal axis of the catheter from the body portion 622. The clamping portion located on the opposite side includes a clamping surface for clamping the catheter 108. A second clamping portion 628 attached or integral with the second body portion 624 is located on the opposite side of the central longitudinal axis of the catheter from the body portion 624. The clamping portion located on the opposite side includes a clamping surface for clamping the catheter 108.

The clamping surfaces are opposed to each other such that when the body portions (622 and 624) slide relative to each other by the force of the force generating member 608, the opposed clamping surfaces move closer to each other to clamp down on the catheter 108. By applying a force that opposes and is greater than the force of the force generating member 608, the clamping surface can be separated as the body portions move back closer to a fully aligned position, wherein the fully aligned position refers to one being on top of the other.

FIGS. 11A-D are schematic illustrations of example catheter clamps where a needle has been removed from the lumen of the catheter 108 and the clamping surfaces (202 and 204) have moved to a position which clamps down on the catheter 108 to reduce or eliminate blood flow through the catheter. Each of the example catheters illustrated in FIGS. 11A-D include a pivot location 702. The pivot location 702 provides a location of rotational motion that allows the clamping surfaces to move both towards and away from each other.

Referring to FIG. 11A, for example, the surfaces 202 and 204 have rotated into a position where they clamp the catheter so that blood flow through the catheter lumen is reduced or eliminated. A force is provided by the force generating member 704 which biases the surfaces, and rotation about the pivot location, towards each other and towards the position where the catheter is clamped. In this regard, the force generating member optionally includes inherent tensile properties that tend to bias the surfaces (202 and 204) towards each other into the clamped position.

The example clamp of FIG. 11A further includes two lever arms 712 that can be manipulated by an operator of the device to provide a force about the location of rotational movement that opposes the biasing force of the force generating member 704. By applying a force to the lever arms 712, the operator can cause the surfaces 202 and 204 to move away from each other and away from the central longitudinal axis of the catheter 108. This can result in opening of the clamp to a position where it can be removed from the catheter, placed on the catheter, and/or to where blood is able to flow through the catheter. Similarly, an operator may optionally press inwards on opposing portions of the force generating member 704 to cause opening of the clamping surfaces.

Now referring to FIG. 11B, there is no needle in the lumen of the catheter 108, and the surfaces 202 and 204 have rotated into a position where they clamp the catheter 108 so that blood flow through the catheter lumen is reduced or eliminated. A force is provided by the force generating member 706 which biases the surfaces (202 and 204), and rotation about the pivot location (702), towards each other and towards the position where the catheter is clamped. In this regard, the force generating member optionally includes inherent tensile properties that tend to bias the surfaces (202 and 204) towards each other into the clamped position.

As discussed throughout, the clamped position may reduce or eliminate blood flow through the catheter. An operator may optionally press inwards on opposing portions of the force generating member 706 to cause opening of the clamping surfaces. This can result in opening of the clamp to a position where it can be removed from the catheter, placed on the catheter, and/or to where blood is able to flow through the catheter.

Now referring to FIG. 11C, there is no needle in the lumen of the catheter 108, and the surfaces 202 and 204 have rotated into a position where they clamp the catheter 108 so that blood flow through the catheter lumen is reduced or eliminated. A force is provided by the force generating member 708 which biases the surfaces (202 and 204), and rotation about the pivot location (702), towards each other and towards the position where the catheter is clamped. In this regard, the force generating member optionally includes inherent tensile properties that tend to bias the surfaces (202 and 204) towards each other into the clamped position.

As discussed throughout, the clamped position may reduce or eliminate blood flow through the catheter. An operator may optionally press inwards on opposing portions of the force generating member 708 to cause opening of the clamping surfaces. This can result in opening of the clamp to a position where it can be removed from the catheter, placed on the catheter, and/or to where blood is able to flow through the catheter.

Now referring to FIG. 11D, there is no needle in the lumen of the catheter 108, and the surfaces 202 and 204 have rotated into a position where they clamp the catheter 108 so that blood flow through the catheter lumen is reduced or eliminated. A force is provided by the force generating member 710 which biases the surfaces, and rotation about the pivot location, towards each other and towards the position where the catheter is clamped. In this regard, the force generating member optionally includes inherent tensile properties that tend to bias the surfaces (202 and 204) towards each other into the clamped position.

The example clamp of FIG. 11A further includes two lever arms 714 that can be manipulated by an operator of the device to provide a force about the location of rotational movement that opposes the biasing force of the force generating member 710. By applying a force to the lever arms 714, the operator can cause the surfaces 202 and 204 to move away from each other and away from the central longitudinal axis of the catheter 108. This can result in opening of the clamp to a position where it can be removed from the catheter, placed on the catheter, and/or to where blood is able to flow through the catheter. Similarly, an operator may optionally press inwards on opposing portions of the force generating member 710 to cause opening of the clamping surfaces.

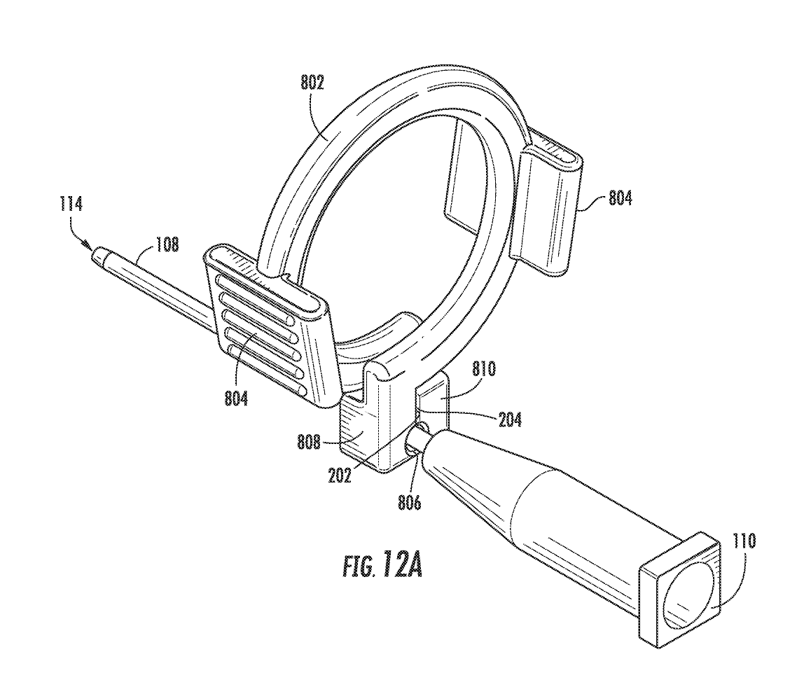

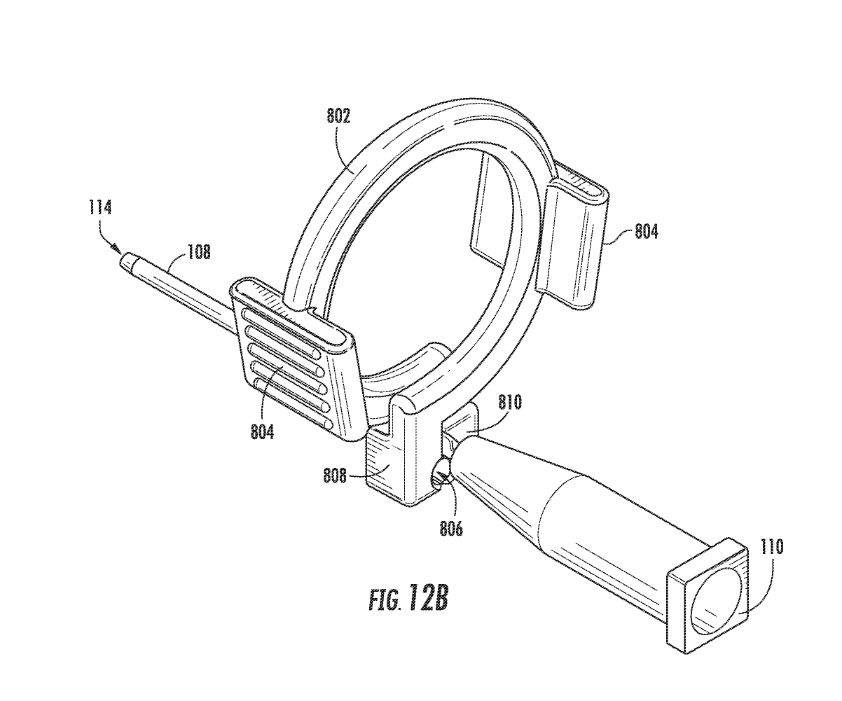

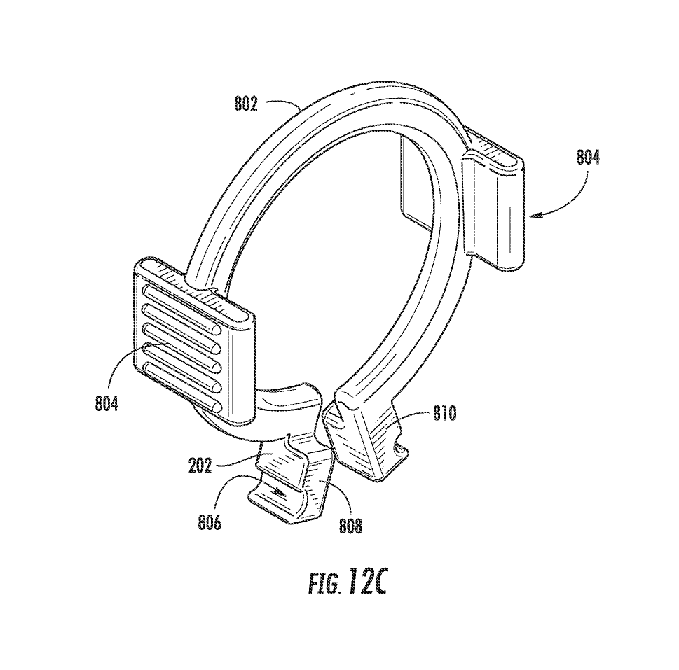

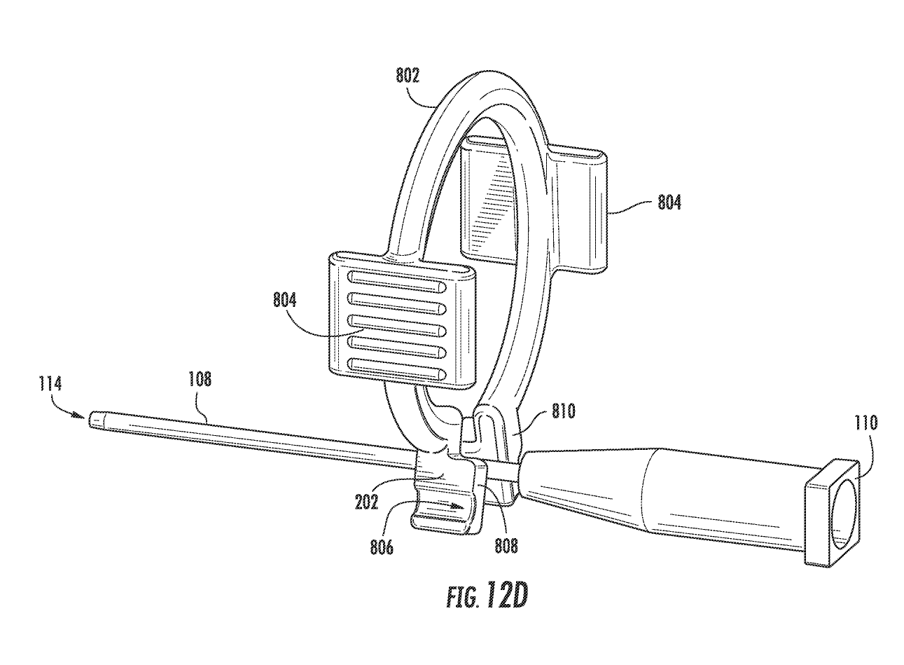

FIGS. 12A-D are schematic illustrations of an example clamping device. The clamping device is optionally positioned on a catheter when a needle is threaded through the lumen of the catheter. Upon removal of the needle, opposed clamping surfaces of the catheter clamp move relative to each other to narrow a space between the surfaces. Because a portion of the catheter without the needle is positioned in the space between the surfaces, and because the force applied to the catheter by the clamping surfaces is greater than the hoop strength of the catheter, once the needle is removed, the lumen of the catheter is narrowed or occluded by the crimping action of the clamping surfaces. This crimping action results in a reduction or elimination of blood flow through the catheter lumen when the catheter is operatively positioned in the vasculature of a patient.

FIG. 12A shows a pre-clamped position where a portion of the catheter 108 is located in a protective tubular structure 806 defined by a first clamping body 808 and a second clamping body 810 of the catheter clamp. In this position, the clamping surfaces (202 and 204), also located on the clamping bodies, are clamped together and are located above the tubular structure 806, which is defined below the clamping surfaces by lower portions of the clamping bodies. The shape or circumference of the tubular structure optionally is sized to fit a variety of catheter types and sizes.

Although the internal walls of the tubular structure 806 may exert a force on the catheter, for example, a force sufficient to hold the catheter in position relative to the clamp, when the catheter 108 is in the protective tubular structure 806 the clamp does not exert a continued, substantial force on the catheter. For example, the force applied to the catheter by the walls of the tubular structure optionally hold the catheter in place relative to the clamp, but do not cause crimping of the catheter if the needle is removed from the catheter lumen, as shown, or if the needle is in place but does not produce pressure on the catheter to cause significant structural changes to the catheter over time.

The clamping bodies (808 and 810) are attached or are integral with a biasing member 802. The biasing member 802 optionally has a spiral shape such that the location where the second clamping body 810 is attached to or integral with the biasing member 802 is distal to the location where the first clamping body 808 is attached to or integral with the biasing member 802. The biasing member has internal tensile properties that urge it to assume an enlarging spiral radius. Thus the spiral is biased towards outward expansion. This biasing force urges the clamping bodies to move in opposite directions relative to each other when positioned around the catheter as shown in FIG. 12A. As shown in FIG. 12A, however, the motion created by the biasing force is stopped by the approximation of the catheter-side surfaces of the clamping bodies, including, for example, the clamping surfaces 202 and 204 and other portions of the clamping body that define the protective tube 806.

The clamping device optionally includes surfaces 804 designed for an operator to pinch to oppose the force of the biasing member 802. By pinching the surfaces 804 with sufficient force to overcome the force of the biasing member 802, the operator can open the clamp by causing the clamping bodies (808 and 810) to move in the opposite direction as the move when moved by the biasing member. When the bodies (808 and 810) are moved apart a sufficient amount the catheter clamp is in a configuration that allows it to optionally be removed from the catheter, positioned on a catheter, or moved relative to the catheter.

The clamping bodies can also be moved apart to a sufficient extent to create a slot or space between the clamping surfaces 202 and 204 of a size that allow the location of a catheter of desired outer diameter therein the slot or space. Then when the operator reduces or eliminates the applied force that opposes the natural force of the biasing member, the clamping surfaces 202 and 204 move back towards each other to clamp against the catheter. If the needle is still in place within the catheter, the clamping surfaces 202 and 204 will rest against the catheter, but will not be able to crimp the catheter lumen due to the stent-like effect of the needle. When the needle is removed, however, as shown in FIG. 12B, the stent-like effect is removed and the biasing force of the force generating member 802 is sufficient to overcome the hoop strength of the non-stented catheter. This causes a crimping of the catheter. The crimping reduces or eliminates flow of blood through the lumen of the catheter.

FIG. 12C illustrates the clamping device shown in FIGS. 12A and 12B without a corresponding catheter and in a fully open position. In this position, the biasing force of the spiral biasing member has been allowed to maximally open the spiral radius. This configuration allows the clamping bodies 808 and 810 to fully separate. When the clamping bodies 808 and 810 fully separate, the clamping device optionally transitions from the spiral configuration shown in FIGS. 12A and 12B to a less severe spiral or ring shape.

When removing the clamping device from a catheter, the operator optionally pinches the device at the pinching surfaces to separate the clamping surfaces and then moves the second clamping body 810 distal relative to the first clamping body 808. Similarly, the first clamping body can be moved proximal relative to the second clamping body, or both clamping bodies can be moved with the second body being moved distally and the first being moved proximally. When the clamping surfaces are no longer in alignment for clamping, the pinching pressure may be released thereby allowing the device to expand to the open position shown in FIG. 12C.

The clamp shown in FIG. 12C can also be manipulated for placement onto a catheter. For example, the operator can move the bodies so that the clamping surfaces are not in alignment and such that clamping bodies can slide past one another across the top-to-bottom midline plane of the force generating member 802. The top-to-bottom midline plane is the plane that bisects the force generating member 802 at its peak and extends down along the midline between the first body 808 and second body 810. Then the operator can pinch the device until the clamping bodies slide past each other and cross the top-to-bottom midline plane.

The operator can then reposition the clamping bodies into alignment such that their clamping surfaces may come together to crimp a catheter positioned between the clamping surfaces. Finally, the operator may release the pinch pressure to allow the clamping bodies to come together such that the bodies form the protective tube 806 and such that the clamping surfaces can crimp a catheter positioned there between the clamping surfaces. FIG. 12D shows the catheter clamp in its open position and with a catheter positioned between the clamping bodies before they have been moved into their clamping positions. In this figure, the central axis of the catheter lumen is along the top-to-bottom midline plane of the force generating member 802.

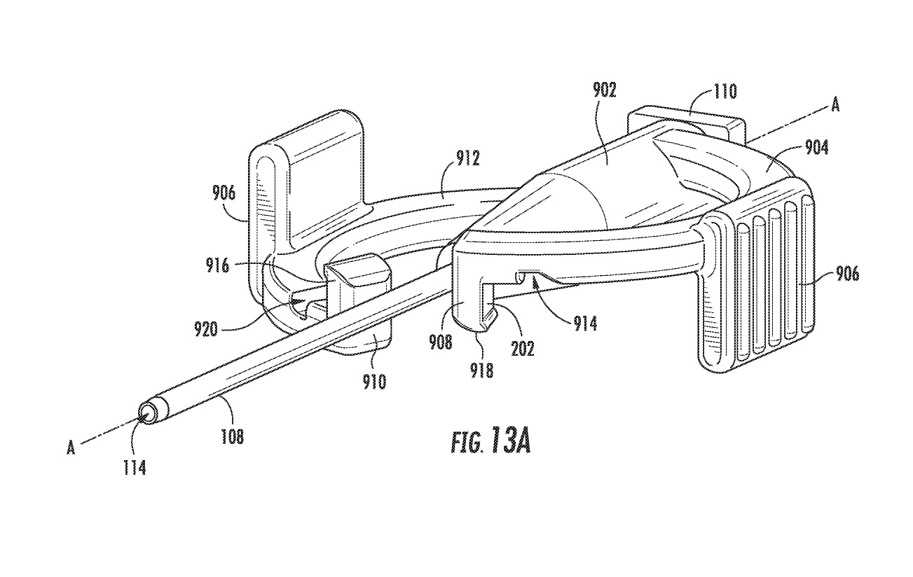

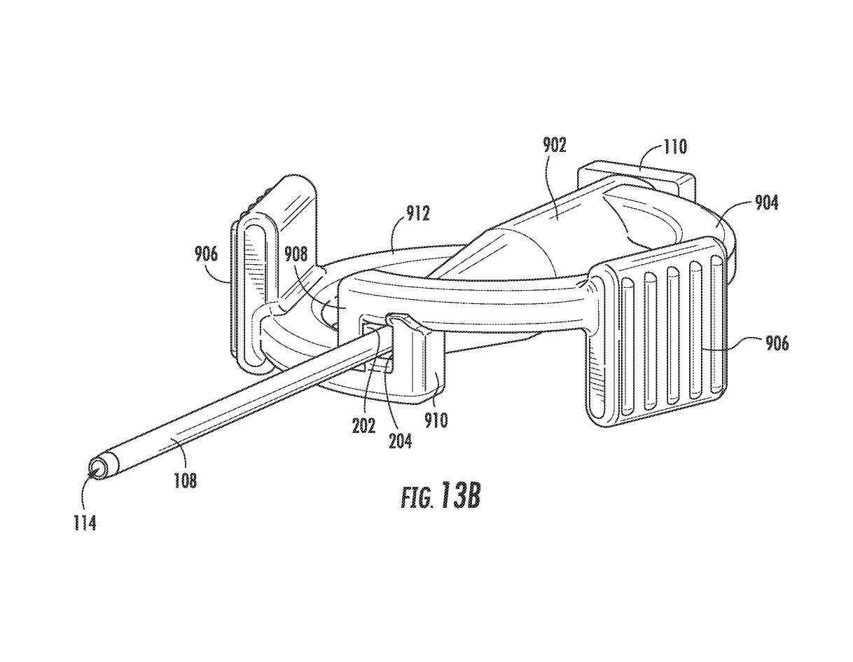

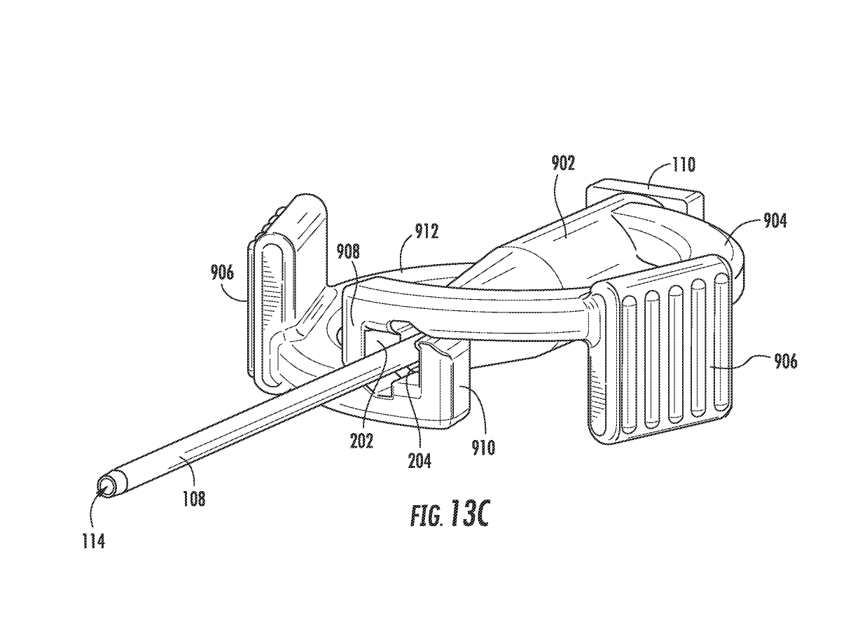

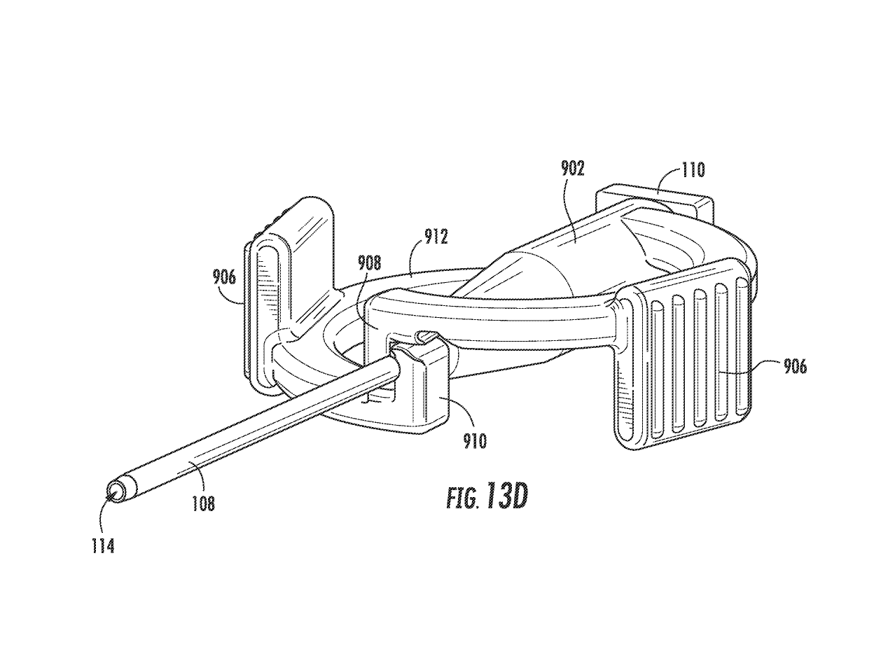

FIGS. 13A-D are schematic illustrations of a combined catheter and catheter clamp device. The device includes a catheter 108 with a distal tip 114 for insertion into the vasculature of a subject. The catheter 108 further includes a hub portion 902 and a proximal portion 110. The distal portion defines an opening for insertion of a guide, such as a needle. As discussed above, a catheter with a needle inserted through the opening defined at the proximal portion 110 is threaded through the catheter lumen and is optionally advanced until the sharp tip of the needle extends distal to the distal catheter portion. The needle, catheter combination can be threaded into a vessel of the subject and the needle can then be removed, leaving the catheter within the vessel to provide direct access to the subject's vascular system. The device shown in FIGS. 13A-D further includes an integral clamping mechanism that can clamp or crimp the catheter upon removal of a needle or intraluminal guide to reduce or eliminate blood flow through the catheter lumen.

The clamping mechanism includes a first clamping body 908 and a second clamping body 910. The first clamping body includes a first clamping surface 202 and the second clamping body includes a second clamping surface 204. The first and second clamping surfaces are moveable relative to one another and can be moved towards one another to clamp a catheter positioned between the surfaces. The first clamping body is connected to, attached or integral with a first arm 904. The second clamping body is connected to, attached, or integral with a second arm 912. The first and second arms are connected to, are attached, or are integral with the catheter hub portion 902. The two arms (912 and 904), combined with the hub portion 902, act as a force generating member for applying the clamping or crimping force to the catheter 108.

FIG. 13A illustrates the device with the arms outwardly extended. In this position, the arms resist being repositioned towards the catheter such that their inherent tensile properties urge each arm outward. Each arm optionally includes a surface 906 that may be pinched by an operator to move the clamping bodies closer and across the central axis (A-A) of the catheter lumen such that the first clamping body 908, including the first clamping surface 202 can be positioned on the opposite side of the central lumen axis from the first arm 904. Similarly, the second clamping body 910, including the second clamping surface 204, can be positioned opposite side of the central lumen axis from the second arm 912. This configuration is shown, for example, in FIGS. 13B-D. In the configurations of FIGS. 13B-D, the arms seek to extend outward to assume the position shown in FIG. 13A. When the first clamping portion 908 and the second clamping portion 912 are aligned, however, the arms are prevented from extending fully outward as portions of the clamping bodies rest against each other.

As shown in FIGS. 13A and 13B, the first clamping body can include a protrusion 918 configured to rest in a corresponding notch 920 in the second clamping body. Similarly, the second clamping body can include a protrusion 916 configured to rest in a corresponding notch 914 in the first clamping body. When the protrusions are engaged in the corresponding notches the notch or space between the first clamping surface 202 and the second clamping surface 204 is maintained at a width that does not apply a substantial clamping or crimping force on the catheter. For example, as shown in FIG. 13B, the catheter is positioned in the space between the clamping surfaces and the surfaces are not clamping the catheter to an extent that blood flow would be reduced or eliminated through the catheter lumen.

In FIG. 13C, for example, by applying a pinching pressure to the surfaces 906, the protrusions are moved out of their corresponding notches and the width of the space between the clamping surfaces is expanded. Then, as shown in FIG. 13D, the clamping surfaces are allowed to come together to clamp the catheter under the force generated by the arms 912 and 904. If the catheter is inserted in the vasculature of a patient, blood flow through the catheter lumen can be reduced or eliminated by the clamping action of the device. Moreover, if a needle or intraluminal guide device were threaded though the catheter lumen, as the device moved between the configuration shown in FIG. 13C and FIG. 13D, the needle or guide would act as a stent like device to prevent the catheter from being clamped as shown in FIG. 13D. Then upon removal of the needle or guide, the clamping surfaces would automatically narrow further to clamp the non-stented catheter portion positioned in the slot or space between the clamping surfaces.

Also provided are methods of reducing fluid flow, such as blood, through a catheter lumen. For example, a catheter clamping device is placed on an intravenous catheter before the catheter is inserted in a vein. Upon confirmation of the venous access, as the metal needle inside the catheter is pulled out, the spring loaded clamp shuts the plastic catheter and thus preventing leakage of the patient's blood, thus eliminating its exposure. Upon connection to the IV line, the clamp is taken off of the catheter.

Thus, the methods comprise placing a catheter with a needle positioned in its lumen into a vessel or other fluid containing space in a subject. A catheter clamp is positioned over the catheter such that at least a portion of the catheter is located in a slot defined by two opposed surfaces of the device that are moveable relative to one another. The needle is removed from the catheter to permit access to the vessel or fluid containing space and upon removal, the surfaces of the clamp move relative to one another to prevent or reduce fluid flow through the catheter.

An IV line or other therapeutic device or unit is positioned in communication with the catheter and once so positioned, the clamp is removed to allow fluid communication between the intravascular space or fluid containing space and the IV line or therapeutic device. The methods thereby reduce or prevent blood or fluid spillage from the catheter while establishing access to fluid filled spaces in the subject, such as the intravascular space.

The devices and methods described herein can be manufactured inexpensively and provide a barrier to the blood leaking through an intravenous catheter. In addition to the intravenous catheters, this device and methods can be used during the placement of the intra-arterial catheters as well as any other catheters placed for medical or surgical reasons.

The devices and methods do not require a change of the IV placement technique. Moreover, the devices and methods can be used for a wide variety of catheter sizes ranging from 24 gauge all the way up to the 14 gauge. The force that the opposed surfaces exert on the catheter can be varied depending on factors including the catheter size and the desired reduction of blood flow (e.g. from minimal reduction to prevention).

A wide variety of materials can be used to construct the device. For example, medical grade plastic can be used for single use, disposable clamps, whereas materials such as steel or titanium can be used if a reusable clamp is desired after sterilization.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A device for reducing or preventing fluid flow through a catheter lumen, comprising:
a. a first clamping surface;
b. a second clamping surface opposed to the first clamping surface, wherein the surfaces at least partially define a slot configured to accept a portion of the catheter;
wherein the first and second surfaces are moveable relative to one another to narrow the slot, and wherein the narrowed slot at least partially occludes the catheter lumen thereby reducing or preventing fluid flow through the catheter.

2. The device of claim 1, wherein the fluid is blood.

3. The device of claim 1, wherein the catheter positioned in the slot has a needle threaded through its lumen.

4. The device of claim 3, wherein the first and second surfaces move relative to each other to narrow the slot and to at least partially occlude the catheter lumen upon removal of the needle from the catheter lumen.

5. The device of claim 1, further comprising an actuator configured to move the surfaces relative to each other.

6. The device of claim 5, wherein the actuator is a spring.

7. The device of claim 6, wherein a first end of the spring is coupled to the first surface and wherein a second end of the spring is coupled to the second surface and wherein force generated by the spring causes movement of the surfaces relative to each other to narrow the slot and to at least partially occlude the lumen.

8. The device of claim 1, further comprising first and second clamping bodies, the first clamping body comprising the first clamping surface and the second clamping body comprising the second clamping surface.

9. The device of claim 8, further comprising a force generating member connected to each clamping body.

10. The catheter of claim 9, wherein the force generating member biases the first and second clamping surfaces towards one another with sufficient force to deform the portion of the catheter that is positioned in the space between the clamping surfaces.

11. A method of reducing or preventing blood flow through a catheter located in a blood vessel of a subject, comprising:
a. positioning the catheter having a needle threaded through its lumen in the slot of the device of claim 1;
b. removing the needle from the catheter lumen, wherein the first and second surfaces move relative to each other to narrow the slot upon removal of the needle from the catheter lumen to at least partially occlude the catheter lumen thereby reducing or preventing blood flow though the catheter.

12. The method of claim 11, wherein the vessel is a vein or artery.

13. A catheter, comprising:
a. a flexible tubular portion for placement into the vasculature of a subject,
b. a clamping device having a first clamping surface and a second clamping surface opposed to the first clamping surface, wherein the surfaces at least partially define a space configured to accept a portion of the flexible tubular portion of the catheter; and
wherein the first and second surfaces are moveable relative to one another to narrow the space, and wherein the narrowed space at least partially occludes the catheter lumen at a location along the tubular portion thereby reducing or preventing fluid flow through the catheter.

14. The catheter of claim 13, wherein the clamping device further comprises a first arm with a distal end connected to a first clamping body, the first clamping body comprising the first clamping surface.

15. The catheter of claim 13, wherein the clamping device further comprises a second arm with a distal end connected to a second clamping body, the second clamping body comprising the second clamping surface.

16. The catheter of claim 13, wherein catheter includes a hub portion, distal to the tubular portion.

17. The catheter of claim 16, wherein each arm further comprise a proximal end that is connected to the hub portion.

18. The catheter of claim 17, wherein the first clamping surface is located opposite the central midline axis of the catheter from the connection of the proximal end of the first arm with the hub.

19. The catheter of claim 17, wherein the second camping surface is located opposite the central midline axis of the catheter from the connection of the proximal end of the second arm with the hub.

20. The catheter of claim 18, wherein the arms bias the first and second clamping surfaces towards one another with sufficient force to deform the portion of the flexible tubular portion of the catheter that is positioned in the space between the clamping surfaces.

21. The catheter of claim 20, wherein the deformation of the flexible tubular portion is sufficient to reduce or eliminate blood flow through the catheter.
